(19) United States
(12) Patent Application Publication
(10) Pub. No.: US 2011/0237848 A1
(43) Pub. Date: Sep. 29, 2011

(54) PROCESS AND APPARATUS FOR PRODUCING ETHYLENICALLY UNSATURATED HALOGENATED HYDROCARBONS

(76) Inventors: Michael Benje, Bad Soden (DE); Peter Kammerhofer, Burgkirchen (DE); Klaus Krejci, Burghausen (DE); Rainer Kampschulte, Burghausen (DE); Helmut Grumann, Perach (DE)

(21) Appl. No.: 12/998,173

(22) PCT Filed: Sep. 3, 2009
(86) PCT No.: PCT/EP2009/006382

§ 371 Date: Jun. 7, 2011

(30) Foreign Application Priority Data
Sep. 26, 2008 (DE) 10 2008 049 262.0

(51) Int. Cl. C07C 17/00; B01J 19/00

(52) U.S. Cl. 570/226; 570/216; 422/105

(57) ABSTRACT

The invention is directed to a process and to an apparatus for saving fuel in furnaces for thermal dissociation of halogenated aliphatic hydrocarbons, especially of 1,2-dichloroethane, using chemical dissociation promoters or physical measures which initiate the dissociation reaction.

The initiation of the dissociation reaction lowers the temperature level in the reaction mixture with the same conversion. This can also lower the mean firing chamber temperature and save fuel.

In a preferred process variant, flue gas leaving the convection zone of the dissociation oven is analyzed and its dew point is calculated. The dew point of the flue gas or the conversion of the dissociation reaction serves as command parameter for the intensity of the physical measure for initiation and/or for the amount of the chemical dissociation promoter added and/or for the amount of fuel.

In a further preferred process variant, the latent heat content of the flue gas is used to preheat the burner air or other media.

### CLAIM FOR PRIORITY

This substitute specification is submitted as a national phase entry of International Patent Application No. PCT/EP2009/006382 (International Publication No. WO 2010/034395), filed Sep. 3, 2009, entitled “Method and Device for Producing Ethylenically Unsaturated Halogenated Hydrocarbons” which claims priority to German Patent Application No. DE 10 2008 049 262.0, filed Sep. 26, 2008 and is entitled, “Verfahren Und Vorrichtung Zur Herstellund Von Ethylenisch Ungesattigten Halogenierten Kohlenwasserstoffen”. The priorities of International Patent Application No. PCT/EP2009/006382 and German Patent Application No. DE 10 2008 049 262.0 are hereby claimed and their disclosures incorporated herein by reference in their entireties.

### DESCRIPTION

The present invention relates to a particularly economical process and an apparatus suitable therefor for preparing ethylenically unsaturated halogen compounds by thermal dissociation of halogenated aliphatic hydrocarbons, in particular the preparation of vinyl chloride by thermal dissociation of 1,2-dichloroethane. The invention is directed at saving fuel in dissociation furnaces for carrying out these dissociation reactions.

The invention is described below by way of example for the production of vinyl chloride (hereinafter referred to as VCM) by thermal dissociation of 1,2-dichloroethane (hereinafter referred to as EDC), but can also be used for the preparation of other ethylenically unsaturated halogen compounds.

Terminology used herein is given its ordinary meaning unless otherwise stated herein.

### BRIEF DESCRIPTION OF THE DRAWINGS

The invention is described in detail below with reference to the drawings wherein like numbers designate similar parts and wherein:

FIG. 1 is a schematic of a reactor for producing ethylenically unsaturated halogenated hydrocarbons for halogenated aliphatic hydrocarbons as described herein.

FIG. 1A is a schematic of an older style of reactor for producing ethylenically unsaturated halogenated hydrocarbons for halogenated aliphatic hydrocarbons retrofitted to accept the invention as described herein.

FIG. 2 is a schematic of the integration of the reactor of FIG. 1 into a system for producing ethylenically unsaturated halogenated hydrocarbons for halogenated aliphatic hydrocarbons as described herein.

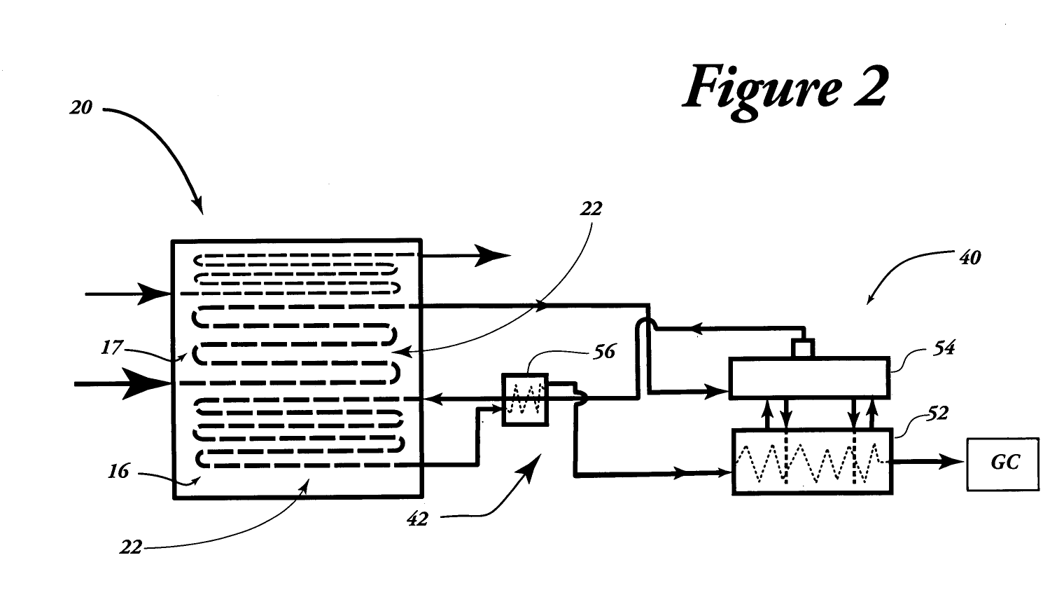

VCM is nowadays prepared predominantly by thermal dissociation of EDC, with the reaction being carried out industrially according to the equation

C₂H₄Cl₂+71 kJ- - - - - >C₂H₃Cl+HCl

in a reaction tube 22 which is in turn located in a gas- or oil-heated furnace 20.

The reaction is usually allowed to proceed to a conversion of 55-65%, based on the EDC used (hereinafter feed EDC). The temperature of the reaction mixture leaving the furnace 20 (hereinafter furnace exit temperature) is about 480-520° C. The reaction is carried out under superatmospheric pressure. Typical pressures at the furnace inlet are about 13-30 bar abs. in present-day processes.

At higher conversions and, resulting therefrom, a higher partial pressure of VCM in the reaction mixture, VCM is increasingly converted under the reaction conditions into subsequent products such as acetylene and benzene which in turn are precursors of carbon deposits. The formation of carbon deposits makes shutdown and cleaning of the reactor at regular intervals necessary. In view of this, a conversion of 55%, based on the EDC used, has been found to be particularly advantageous in industrial practice.

The majority of processes employed at present operate using cuboidal furnaces 20 in which the reaction tube 22 is arranged centrally as a serpentine tube made up of horizontal tubes 22a, 22s, 22b arranged vertically above one another, with the serpentine tube being able to have a single or double configuration. In the case of a single configuration, the tubes 22a, 22s, 22b can either be aligned or offset. The furnaces 20 are heated by means of burners 26, 28 which are arranged in rows in the furnace walls 24. The transfer of heat to the reaction tubes 22a, 22s, 22b occurs predominantly by wall and gas radiation but also convectively via the flue gas 38 formed in heating by means of burners 26. The dissociation of EDC is sometimes also carried out in other types of furnace 20 having a different arrangement of the reaction tubes 22a, 22s, 22b and the burners 26.

The part of the furnace 20 in which the burners 26 and the reaction tube 22a are arranged and the dissociation reaction takes place is referred to as the radiation zone 16. Above the actual reaction tubes 22b and upstream of the radiation zone 16 viewed in the flow direction of the reaction mixture there are usually further rows of unfinned tubes 22s which are composed of tubes arranged horizontally next to one another and largely shield internals located above them (the finned heat exchange tubes 22a of the convection zone 17) against direct radiation from the firing space and, in addition, increase the thermal efficiency of the reaction zone by means of structurally optimized convective heat transfer. In technical language usage, these tubes 22s or rows of tubes are usually referred to as “shock tubes” or “shock zone”.

The invention can in principle be applied to all types of furnace 20 and burner 26, 28 arrangements and also to other ways of heating the reaction.

A typical tube reactor used for the dissociation of EDC comprises a furnace 20 and a reaction tube 22. In general, such a furnace 20 fired by means of a primary energy carrier, e.g. oil or gas, is divided into a radiation zone 16 and a convection zone 17.

In the radiation zone 16, the heat required for the dissociation is transferred to the reaction tube 22 primarily by radiation from the burner-heated furnace walls 24 and the hot flue gas 38.

In the convection zone 17, the energy content of the hot flue gases 38 leaving the radiation zone 16 is utilized by convective heat transfer. In this way, the starting material for the dissociation reaction, e.g. EDC, can be preheated, vaporized or superheated. The generation of steam and/or the preheating of combustion air is likewise possible.

In a typical arrangement as described, for example, in EP 264,065 A1 (incorporated herein by reference in its entirety), liquid EDC is firstly preheated in the convection zone 17 of the dissociation furnace 20 and then vaporized in a specific vaporizer 40 outside the dissociation furnace 20. The gaseous EDC is then fed into the convection zone 17 again and superheated there, preferably in the shock tubes 22s, with the dissociation reaction being able to commence here. After superheating has occurred, the EDC enters the radiation zone 16 where the conversion into vinyl chloride and hydrogen chloride takes place.

The burners 26 are usually arranged in superposed rows on the longitudinal sides and end faces of the furnace 20, with efforts being made by means of the type and arrangement of the burners 26 to achieve very uniform distribution of inward radiation of heat along the circumference of the reaction tubes 22.

The part of the furnace 20 in which the burners 26 and the reaction tubes 22b are arranged and in which the predominant conversion of the dissociation reaction takes place is referred to as the radiation zone 16. At the beginning of the radiation zone 16 and above the actual reaction tubes 22b there are usually further rows of tubes 22s, the tubes 22s of which are preferably arranged horizontally next to one another. These are the shock tubes 22s described above.

For the purposes of the invention, the “reaction zone” is made up of the reaction tubes 22b which are located downstream of the shock zone in the flow direction of the reaction gas and are preferably vertically aligned or offset above one another. The major part of the EDC used is converted into VCM here.

The actual dissociation reaction takes place in the gaseous state. Before entering the reaction zone, the EDC is firstly preheated and then vaporized and possibly superheated. Finally, the gaseous EDC enters the reactor 20 where it is usually heated further in the shock tubes 22s and finally enters the reaction zone where the thermal dissociation reaction commences at temperatures above about 400° C.

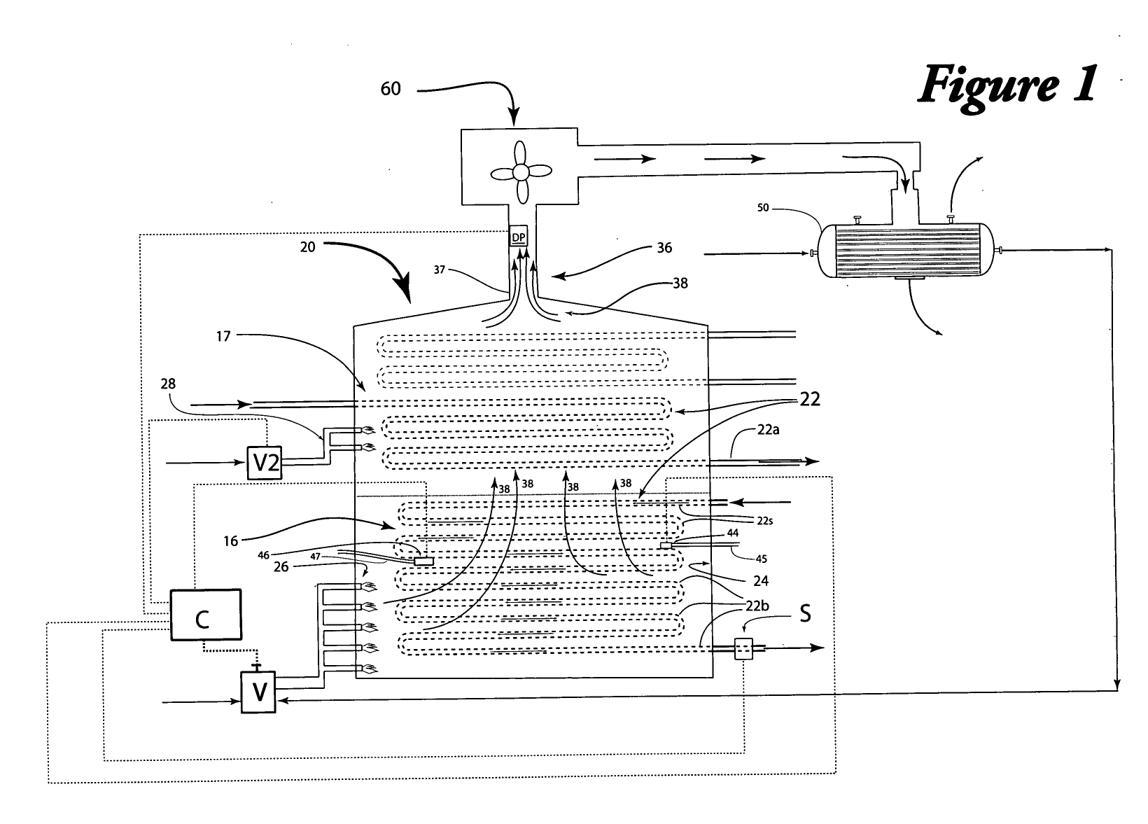

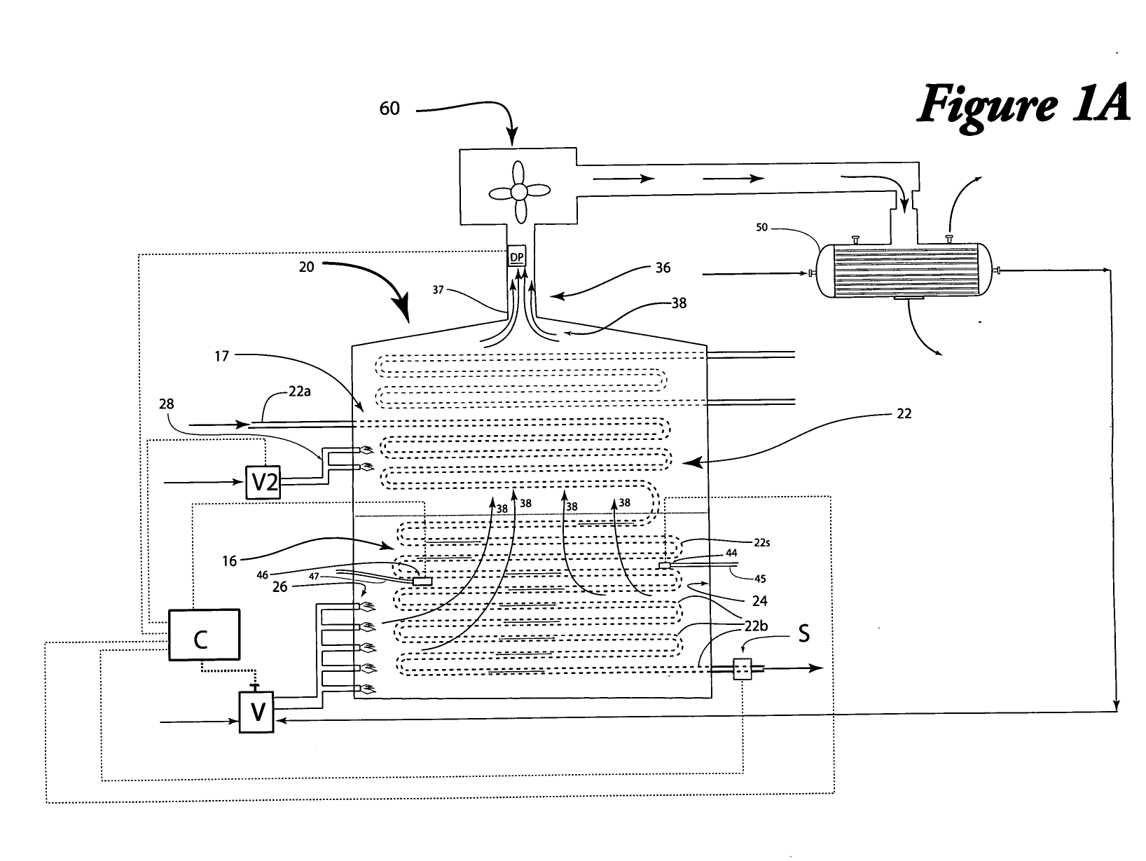

The vaporization of the EDC takes place outside the dissociation furnace 20 in a separate apparatus, viz. the EDC vaporizer 40, in modern plants. The EDC vaporizer 40 is heated by means of steam in some processes. Heating by means of the sensible heat of the reaction mixture leaving the furnace 20 is more economical. In relatively old plants, liquid EDC is introduced into the preheating zone of the furnace 20 and then vaporizes within the furnace 20 as shown in FIG. 1A.

The invention provides a process which comprises vaporization of the feed EDC outside the dissociation furnace 20 by means of a separate apparatus.

In the process of the invention, the sensible heat content of the reaction mixture leaving the dissociation furnace 20 is utilized to vaporize the feed EDC before it enters the dissociation furnace 20, i.e. the EDC vaporizer 40 is heated by means of the hot stream leaving the reactor 20, hereinafter referred to as “dissociation gas”, which is cooled in the process but partial or complete condensation of the dissociation gas is avoided. An apparatus 40 as has been described, for example, in EP 276,775 A2 (incorporated herein by reference in its entirety) has been found to be particularly advantageous for this purpose.

Although the major part of the feed EDC is reacted in the reaction zone, EDC is also converted into VCM in the pipe from the outlet of the dissociation furnace 20 to the inlet into the EDC vaporizer 40, with the reaction adiabatically withdrawing heat from the dissociation gas and the dissociation gas being cooled. This proportion of the total conversion, hereinafter referred to as “after-reaction”, proceeds until entry into the EDC vaporizer 40 where the reaction finally ceases when the temperature drops below a certain minimum. The sum of the volumes of the pipe section from the outlet from the dissociation furnace 20 to the inlet of the EDC vaporizer 40 and the dissociation gas side of the EDC vaporizer 40 itself to the outlet port of the EDC vaporizer is, for the purposes of the invention, referred to as “after-reaction zone” 42.

The heat of the hot flue gas 38 leaving the radiation zone 16 is utilized by convective heat transfer in the convection zone 17 which follows the radiation zone 16 and is physically located above the latter, with the following operations being able to be carried out:
preheating of liquid EDC
vaporization of preheated EDC
heating of heat transfer media
preheating of boiler feed water
generation of steam
preheating of combustion air
preheating of other media (including media extraneous to the process).

Vaporization of EDC in the convection zone 17 is dispensed with in modern plants since in this mode of operation the vaporizer tubes 22 quickly become blocked by carbon deposits, which adversely affects the economics of the process as a result of shortened cleaning intervals.

The physical combination of radiation 16 and convection zone 17 with the associated flue gas chimney 37 is referred to as dissociation furnace 20 by those skilled in the art.

The utilization of the heat content of the flue gas 38, in particular for preheating the EDC, is of central importance for the economics of the process since very complete exploitation of the heat of combustion of the fuel has to be sought.

The reaction mixture leaving the dissociation furnace 20, known as the dissociation gas, contains not only the desired product VCM but also HCl (hydrogen chloride) and unreacted EDC. These are separated off in subsequent process steps and recirculated to the process. Furthermore, the reaction mixture contains by-products which are likewise separated off, worked up and utilized further or recirculated to the process. These relationships are known to those skilled in the art.

The by-products carbon and tar-like substances which are formed over a plurality of reaction steps from low molecular weight by-products such as acetylene and benzene and deposit in the serpentine tubes 22 of the dissociation furnace 20 (and also in downstream apparatuses such as the EDC vaporizer 40) where they lead to a deterioration in heat transfer and, by constricting the free cross section, to an increase in the pressure drop are of particular importance for the process.

Attempts have for a long time been made to increase the space-time yield of the EDC dissociation by means of various measures. These measures have the aim of increasing the amount of product which can be obtained from a given reactor volume and can be divided into:
use of heterogeneous catalysts
use of chemical promoters
other measures (e.g. injection of electromagnetic radiation).

It is generally assumed that the measures proposed hitherto contribute to physical or chemical initiation to provide free chlorine radicals in the reaction space. The thermal dissociation of EDC is a free-radical chain reaction in which the first step is elimination of a free chlorine radical from an EDC molecule:

C₂H₄Cl₂- - >C₂H₄Cl

The high activation energy of this first step compared to the subsequent chain propagation steps is the reason why the dissociation reaction proceeds appreciably only above a temperature of about 420° C.

The use of a heterogeneous catalyst makes elimination of a free chlorine radical from the EDC molecule possible, e.g. by dissociative adsorption of the EDC molecule on the catalyst surface. Very high EDC conversions can be achieved using heterogeneous catalysts. However, decomposition of the VCM and thus carbon formation on the catalyst surface occur on and in the vicinity of the catalyst surface as a result of high local partial pressures of VCM, leading to rapid deactivation of the catalyst. Owing to the frequent regenerations made necessary thereby, heterogeneous catalysts have hitherto not been employed in the large-scale production of VCM.

In the case of physical measures, e.g. irradiation with short-wavelength light, the energy for elimination of the free chlorine radical is provided from an external source. Thus, adsorption of a quantum of short-wavelength light by the EDC molecule provides the energy for elimination of the free chlorine radical:

C₂H₄Cl₂+hν- - - - - >C₂H₄Cl+Cl

where “ν” is the frequency of a photon. When chemical initiators are used, a chlorine atom is eliminated from the EDC molecule by reaction of the EDC with the initiator or the free chlorine radicals are provided by decomposition of the initiator. Chemical initiators are, for example, elemental chlorine, bromine, iodine, elemental oxygen, chlorine compounds such as carbon tetrachloride (CCl₄) or chlorine-oxygen compounds such as hexachloroacetone.

All the measures for initiating the reaction bring about a significant reduction in the temperature level in the reactor at a given conversion or a large increase in the conversion at a given temperature level.

Comprehensive literature is available on the use of catalysts for thermal dissociation of EDC. An example which may be mentioned is EP 002,021 A1.

The high tendency of catalysts to become carbonized and the need for frequent regeneration stand in the way of the use of these in industrial practice.

Physical measures such as injection of electromagnetic radiation into the reaction tube 22a (described, for example, in DE 30 08 848 A1 or DE 29 38 353 A1) have also not found their way into industrial practice despite their suitability in principle. The reasons for this may well be related to safety, since, for example, a pressure-resistant optical window is necessary for input of light. Further physical measures which have been described, for instance injection of a heated gas into the reaction mixture (WO 02/094,743 A2) have also not been used hitherto on an industrial scale.

DE 103 19 811 A1 describes the electromagnetic and photolytic induction of free-radical reactions. In addition, this document describes an apparatus for introducing this energy into a reactor. Although this document mentions the use of dissociation promoters in general terms, no information can be found there about the design and the operation of the reactor used.

The use of chemical promoters is in principle the least technically complicated because it is neither necessary to fill the reactor 20 with catalyst (facilities for filling/emptying and regeneration are required) nor are additional facilities for injection of electromagnetic radiation required. The promoter can be introduced into the feed EDC stream in a simple manner.

Increasing the conversion of the EDC dissociation by addition of halogens or halogen-releasing compounds has been described by Barton et al. (U.S. Pat. No. 2,378,859 A), where the experiments of fundamental importance were carried out at atmospheric pressure in a glass apparatus. Krekeler described, in DE patent No. 857,957, a process for the thermal dissociation of EDC under superatmospheric pressure. Carrying out the reaction at superatmospheric pressure is of fundamental importance for large-scale industrial use since only then is economical fractionation of the reaction mixture possible. This relationship is known to those skilled in the art. Krekeler also recognized the problem of accelerated formation of carbon deposits at high conversions and nominated 66% as a practical upper limit to the conversion. In DE-B-1,210,800, Schmidt et al. describe a process in which operation at superatmospheric pressure is combined with the addition of a halogen. Here, conversions of about 90% are achieved at working temperatures of 500-620° C. Schmidt et al. also stated that the conversion reaches saturation as a function of the amount of halogen added, i.e. that a significant increase in conversion is no longer achieved above a particular amount of halogen added relative to the feed EDC stream.

The simultaneous addition of halogen or other chemical promoters at least two points on the reactor tube 22 has been described by Sonin et al. in DE 1,953,240 A. Here, conversions in the range from 65 to 80% were achieved at reaction temperatures of 250-450° C.

Scharein et al. (DE 2 130 297 A) describe a process for the thermal dissociation of EDC under superatmospheric pressure, in which chlorine is introduced at a plurality of points on the reactor tube 22. Here, conversion of 75.6% (example 1) or 70.5% (example 2) are achieved at a reaction temperature of 350-425° C. This publication also refers to the importance of the ratio of surface area/volume of the reactor and to the importance of the loading of the heating areas (heat flux).

The problem of rapid carbonization of the reactor at high conversions of the dissociation reaction is avoided in a process disclosed by Demaiziere et al. (U.S. Pat. No. 5,705,720 A) by diluting the gaseous EDC entering the reactor with hydrogen chloride. Here, hydrogen chloride is added to the EDC in a molar ratio of from 0.1 to 1.8. At the same time, dissociation promoters can also be added to the mixture of EDC and HCl according to this process. Since the VCM partial pressure is kept low by the dilution with large amounts of HCl, high conversions can be achieved without carbonization of the reactor. However, disadvantages here are the energy input for heating and the subsequent removal of the HCl added for dilution.

Longhini (U.S. Pat. No. 4,590,318 A) discloses a process in which a promoter is introduced into the dissociation gas after exit from the dissociation furnace 20, i.e. into the after-reaction zone. Here, the heat content of the dissociation gas is exploited in order to increase the total conversion of the EDC dissociation. However, this method is inferior to measures for increasing the space-time yield in the dissociation furnace itself since only the heat still present in the dissociation gas stream after exit from the dissociation furnace 20 can be exploited and the usable quantity of heat is limited when the heat of the dissociation gas stream is to be utilized for vaporization of the feed EDC.

Felix et al. (EP 133,690 A1), Wiedrich et al. (U.S. Pat. No. 4,584,420 A) and Mielke (DE 42 28 593 A1) teach the use of chlorinated organic compounds instead of chlorine as dissociation promoters. This makes it possible in principle to achieve the same effects on the EDC dissociation reaction as when using elemental halogens such as chlorine or bromine. However, since these are materials which are frequently not (like chlorine, available in the integrated facility for VCM production, they have to be introduced separately into the process, which in turn is associated with increased costs for procuring them and disposal of the residues.

DE 102 19 723 A1 relates to a process for metered addition of dissociation promoters in the course of preparation of unsaturated halogenated hydrocarbons. This document does not disclose any further details regarding the thermal design of the reactor.

Although the effects of dissociation promoters on the reaction of thermal dissociation of EDC and their main advantages have been known for a relatively long time, the use of dissociation promoters has hitherto not found its way into the commercial production of VCM by thermal dissociation.

This is because all previously disclosed processes aim at increased conversions of the dissociation reaction (at least 65%), although it was recognized early on (DE patent no. 857 957) that a significantly increased tendency for carbon deposits to be formed in the reactor tubes 22 and the after-reaction zone 42 has to be expected above this limit. The increased tendency for formation of carbon deposits, which has hitherto prevented the use of dissociation promoters in industrial practice is due not to the promoters themselves but to a combination of higher VCM partial pressures in the reaction mixture (as occur at conversions above 65%) with high temperatures of the dissociation gas and the interior wall of the reactor tube. This assumption is also supported, in particular, by the results disclosed in U.S. Pat. No. 5,705,720 A where high conversions can be achieved with and without dissociation promoter by dilution of the reaction mixture with relatively large amounts of HCl, without an increased tendency for carbon deposits to be formed occurring. Recently, the process for VCM preparation has been exposed to increased cost pressure as a result of the rising energy costs, especially the rising oil and gas prices. Against the background of the climate debate, it also appears advisable, if technically possible, to reduce the CO₂ output of industrial furnaces.

Even though modern plants for preparing VCM have already been substantially energetically optimized by various measures for heat recovery, a further potential for saving is evident on closer inspection of the reaction profile in the dissociation furnace 20. This consists in the fact that the thermal dissociation only begins noticeably at temperatures significantly above 400° C., and that the heat of reaction therefore has to be supplied at a relatively high temperature level.

Therefore, the amount of heat for the heating of the reaction mixture makes up a proportion of approx. 40% of the total amount of heat absorbed in the radiation zone 16. The problem thus consists in allowing the dissociation reaction to proceed at a lower temperature level and hence saving fuel, and in such a way that the secondary process task of the dissociation furnace 20, the preheating of various media in the convention zone 17 by means of the sensible heat content of the flue gas 38, can still be fulfilled.

It is an object of the present invention to provide a reactor 20 having a fuel consumption which is significantly reduced compared to conventional plants with comparable efficiency of the dissociating process.

A further object of the present invention is to provide a process for the thermal dissociation of halogenated aliphatic hydrocarbons, in which significantly reduced amounts of fuel need to be used compared to conventional processes, but which exhibits a comparable efficiency of the dissociating process.

The invention provides a process for the thermal dissociation of halogenated aliphatic hydrocarbons to form ethylenically unsaturated halogenated hydrocarbons in a reactor 20 which comprises reaction tubes 22a, 22s, 22b running through a convection zone 17 and through a radiation zone 16 located downstream in the flow direction of the reaction gas, which has one or more burners 26 in the radiation zone 16 to heat the reaction tubes 22a, 22s, 22b, and a heating apparatus is provided for the halogenated aliphatic hydrocarbon (“feed”) which is located outside the reactor 20 and is heated by the energy content of the reaction gases leaving the radiation zone 16, comprising the measures
a) a chemical promoter for the thermal dissociation is introduced into the reaction tubes 22 and/or localized energy input to promote the thermal dissociation into the reaction tubes 22 is effected at one or more points within the reactor 20,
b) the amount of the fuel for the burner(s) 26 is reduced to such an extent that the conversion of the dissociation reaction changes by ±20%, preferably ±10%, if at all, compared to operation without the chemical promoter and/or the localized energy input,
c) the temperature of the reaction mixture leaving the reactor 20 is in the range from 400° C. to 470° C., and
d) the molar conversion based on the halogenated aliphatic hydrocarbon used is in the range from 50 to 65%.

In a preferred embodiment of the process of the invention, as additional measure (=measure e1), the dew point of the flue gas 38 is measured at the exit from the convection zone 17 or in the flue gas chimney 37 and this serves as command variable for the regulation of the amount of fuel and/or for the regulation of the amount of the chemical promoter added and/or for the regulation of the intensity of the localized energy input.

DE 22 35 212 A describes an improved measuring instrument for the monitoring of the dew point of flue gases. This does not incite the person skilled in the art to use this instrument in processes for thermal dissociation of saturated halogenated hydrocarbons and especially not in conjunction with the use of dissociation promoters and/or locally limited energy inputs.

In a further preferred embodiment of the process of the invention, as additional measure (=measure e2), the flue gas 38 is condensed in a heat exchanger and the waste heat of the flue gas 38 is utilized to preheat the burner air.

In the process variant comprising measure e2), the heat from the cooling of the flue gas 38 below its dew point and the heat of condensation of the flue gas 38 are utilized.

In the case of measure e2), the heat exchange is effected preferably at the exit of the flue gas from the convection zone 17.

The process of the invention may comprise measures e1) or e2), or a combination of measures e1) and e2).

The process of the invention preferably comprises measure e1).

Measure e1) is employed especially in the case of fuels with moderate or high proportions of acid-forming components. However, this measure can also be used in the case of fuels with a low proportion of acid-forming components.

Measure e2) is employed especially in the case of fuels with a low proportion of acid-forming components. However, this measure can also be used in the case of fuels with moderate or high proportions of acid-forming components.

In one embodiment, the invention further provides an apparatus for the thermal dissociation of halogenated aliphatic hydrocarbons to form ethylenically unsaturated halogenated hydrocarbons, which comprises a reactor 20 which comprises reaction tubes 22a, 22s, 22b running through a convection zone 17 and through a radiation zone 16 located downstream in the flow direction of the reaction gas, with one or more burners 26 being provided in the radiation zone 16 to heat the reaction tubes 22a, 22s, 22b, and which comprises a heating apparatus 40 for the halogenated aliphatic hydrocarbon (“feed”) which is located outside the reactor 20 and is heated by the energy content of the reaction gases leaving the radiation zone 16, comprising the elements:
A) means 44 of introducing chemical promoters for the thermal dissociation into the reaction tubes 22 and/or means 46 of introducing localized energy to promote the thermal dissociation at one or more points on the reaction tubes 22,
B) means of introducing the fuel for the burner(s) 26,
C) means DP of determining the dew point of the flue gas 38 at the exit 36 from the convection zone 17 or in the flue gas chimney 37 or means GC for determining the molar conversion of the dissociation reaction, and
D) means of regulating the amount of fuel and/or for regulating the amount of the chemical promoter added and/or for regulating the intensity of the localized energy input, the dew point of the flue gas 38 at the exit 36 from the convection zone 17 or in the flue gas chimney 37 or the molar conversion of the dissociation reaction serving as command variable for the regulation.

In a further embodiment, the invention further provides an apparatus for the thermal dissociation of halogenated aliphatic hydrocarbons to form ethylenically unsaturated halogenated hydrocarbons, which comprises a reactor which comprises reaction tubes 22a, 22s, 22b running through a convection zone 17 and through a radiation zone 16 located downstream in the flow direction of the reaction gas, with one or more burners 26 being provided in the radiation zone 16 to heat reaction tubes 22a, 22s, 22b, and which comprises a heating apparatus for the halogenated aliphatic hydrocarbon (“feed”) which is located outside the reactor 20 and is heated by the energy content of the reaction gases leaving the radiation zone 16, comprising the above-defined elements A), B), C) and D), and additionally:
E) at least one heat exchanger 50 for recovering waste heat from the condensation of the flue gas 38 for preheating the combustion air.

In a preferred embodiment of the inventive apparatus, the means GC of determining the molar conversion of the dissociation reaction is mounted downstream of the exit of the dissociation gas from the heating apparatus 40 for the halogenated aliphatic hydrocarbon or at the top of a quench column.

It has been found that, surprisingly, the fuel consumption of a dissociation furnace 20 can be reduced considerably with the same efficiency of the dissociation process when a chemical promoter is added or an equivalent physical measure for initiating the dissociation reaction, for example the input of high-energy electromagnetic radiation, is used at one or more points in the reaction zone 22b. In this case, the amount of fuel to the oven 20 can be reduced to such an extent that the conversion of the dissociation reaction is unchanged compared to the starting state. At the same time, the dew point of the flue gas 38 can be determined at the exit from the convection zone 17 and used as command variable for the regulation of the amount of fuel or for the regulation of the amount of a dissociation promoter added or the intensity of another measure for initiating the dissociation reaction or for the simultaneous regulation of these two parameters. Alternatively, it is also possible to use the conversion of the dissociation reaction as command variable in order to control the regulation of the amount of fuel and/or the regulation of the amount of a dissociation promoter added and/or the intensity of another measure for initiating the dissociation reaction.

The consumption of fuel of a dissociation furnace 20 at a given efficiency of the dissociating process can likewise be reduced considerably if, in addition to adding the chemical promoter or carrying out an equivalent physical measure for initiating the dissociation reaction, and reducing the amount of fuel to the furnace 20 with no change in the conversion of the dissociation reaction compared to the starting state, the waste heat present in the flue gas 38 is recovered and utilized for preheating the combustion air.

The introduction of chemical promoters for the thermal dissociation can be effected at any points. The promoter can be added to the feed, preferably the gaseous feed. The promoter is preferably introduced into the shock tubes 22s or in particular reaction tubes 22b in the radiation zone 16.

The localized energy input to promote the thermal dissociation is effected into the reaction tubes 22a, 22s, 22b at one or more points within the reactor 20.

The process of the invention is described by way of example for the EDC/VC system. It is also suitable for preparing other halogen-containing unsaturated hydrocarbons from halogen-containing saturated hydrocarbons. In all these reactions, the dissociation is a free-radical chain reaction in which not only the desired product but also undesirable by-products which on long-term operation lead to carbon deposits in the plants are formed. Preference is given to the preparation of vinyl chloride from 1,2-dichloroethane.

For the purposes of the present description, “localized energy input into the reaction tubes to promote the thermal dissociation” refers to physical measures which are able to initiate the dissociation reaction. Such measures can be, for example, injection of high-energy electromagnetic radiation or local introduction of thermal or nonthermal plasmas, e.g. hot inert gases.

Means 44 of introducing chemical promoters for the thermal dissociation together with the halogenated aliphatic hydrocarbon into the reaction tubes in the radiation zone 16 are known to those skilled in the art. These are generally feed lines 45 which allow introduction of predetermined amounts of chemical promoters into the feed gas stream.

Means 44 of introducing chemical promoters for the thermal dissociation at one or more points of the radiation zone 16 into the reaction tubes 22a, 22s, 22b are likewise known to those skilled in the art. These are generally likewise feed lines 45 which allow the introduction of predetermined amounts of chemical promoters into the reaction tubes 22a, 22s, 22b at the level of the radiation zone 16. These feed lines 45 can have nozzles at the reactor end. Preference is given to one or more of these feed lines 45 opening into the tubes 22b in the first third, viewed in the flow direction of the reaction gas, of the radiation zone 16.

Means 46 of introducing localized energy into the reaction tubes 22a, 22s, 22b at one or more points in the radiation zone 16 to form free radicals are likewise known to those skilled in the art. These can likewise be feed lines 47 which may have nozzles at the reactor end and via which thermal or nonthermal plasma is introduced into the reaction tubes 22b at the level of the radiation zone 16; or they can be windows via which electromagnetic radiation or particle beams are injected into the reaction tubes 22b at the level of the radiation zone 16. Preference is given to one or more of these feed lines 47 opening into the tubes in the first third, viewed in the flow direction of the reaction gas, of the radiation zone 16; or the windows for injection of the radiation being installed in the first third.

Ways of selecting the amount of the chemical promoter and/or the intensity of the localized energy input into the reaction tubes 22b to form free radicals are likewise known to those skilled in the art. These are generally regulating circuits in which a command variable is used to regulate the amount or intensity. As command variable, according to one variant of the present invention, the dew point of the flue gas 38 at the exit from the convection zone 17 is used.

The above-described combination of measures or features greatly reduce the consumption of fuel, compared to conventional processes or apparatuses, with no change in the efficiency of the dissociating process.

The process of the invention and the apparatus of the invention are illustrated hereinafter with the addition of a chemical promoter; however, the illustration applies equally to the application of physical measures, for example the incidence of electromagnetic radiation of suitable wavelength. Analogously to the variation of the amount of promoter, for example, the intensity of the electromagnetic radiation can be varied.

The addition of the promoter reduces the proportion of sensible heat in all of the heat absorbed in the radiation zone 16 in favor of the heat of reaction. As a result, the conversion of the reaction rises with the same heating of the furnace 20.

The increase in yield is a function of the amount of promoter metered in up to a certain limit. Above this limit, even the addition of further promoter cannot achieve a further increase in yield. This effect was described, for example, in DE-B-1,210,800.

If the amount of fuel is now reduced, the conversion of the reaction declines again, but the amount and the temperature of the flue gas 38 also do so at the same time. In this context, it should be ensured that the heat content of the flue gas 38 is still sufficient to be able to perform the preheating of process media in the convection zone 17. On the other hand, the temperature must not be lower than the dew point of the flue gas 38 after exit from the convection zone 17 or in the chimney 37, in order to prevent condensation and the associated corrosive attack; or the temperature is deliberately lower than the dew point of the flue gas 38 as a result of the use of one or more heat exchangers 50, and the condensate is removed from the heat exchanger(s) 50.

In one process variant, according to the invention, the dew point of the flue gas 38 is determined. This can be done discontinuously or preferably continuously. The dew point can be determined by determining the water content of the flue gas 38; these analysis values in turn can be used to calculate the dew point of the flue gas. The dew point is used as command parameter for the regulation of the amount of fuel or of the amount of promoter or of the intensity of the local energy input, or more than one of these parameters.

For better exploitation of the heat content of the flue gas, the following measures may additionally be provided:
thermal insulation of the chimney 37
optional additional trace heating of the chimney 37
measurement of the internal wall temperature of the chimney 37
use of a flue gas (chimney) blower 60
use of heat exchanger(s) 50 to cool the flue gases 38 below the dew point.

If the internal wall temperature of the chimney 37 is measured, the value thereof, depending on the dew point of the flue gas 38, can also be used as command variable for the setting of the flue gas 38 temperature at the exit 36 from the convection zone.

In this way, in existing plants, design reserves of the heat exchanger area in the convection zone 17 can be exploited. New plants can be designed with dimensions for better heat exploitation from the outset by virtue of appropriate dimensions of the heat exchange areas in the convection zone 17. The process of the invention can thus be employed in new plants, in which case the heat exchange area in the convection zone 17 is designed with dimensions for lower temperature differences. In existing plants, area reserves of the convection zone 17 can be exploited, or these can be increased by installing additional area, as far as possible.

In a further preferred variant of the process of the invention, the flue gas 38 is extracted by means of a flue gas blower 60 after leaving the convection zone 17 and is passed through one or more heat exchangers 50 where it is condensed. The waste heat is utilized for heating the burner air or other media. The condensate formed is, if appropriate, worked up and discharged from the process. The remaining gaseous constituents of the flue gas 38 are, if appropriate, purified and released into the atmosphere.

Particular preference is given to a process in which the flue gas 38 to be cooled to below the dew point is introduced in a downward direction from above into the heat exchanger 50 provided for this purpose, after cooling leaves the heat exchanger 50 in the upward direction and the condensate formed can freely runoff downward from the heat exchanger 50 and is thus completely separated off from the flue gas stream 38.

When carrying out the process of the invention, at one or more points on the shock tubes 22s or the tubes in the reaction zone 22b, electromagnetic radiation of a suitable wavelength or a particle beam is radiated in or a chemical promoter is added or a combination of these measures is undertaken. In the case of the addition of a chemical promoter, the addition can also be into the feed line 45 for the feed, preferably for the gaseous feed, in particular into the EDC downstream of the EDC vaporizer 40, on entry into the dissociation furnace 20, but very particularly preferably in the radiation zone 16.

The localized energy input to form free radicals is preferably effected by electromagnetic radiation or particle beams; the electromagnetic radiation is here particularly preferably ultraviolet laser light.

In the case of addition of a chemical promoter, the use of elemental halogen, in particular elemental chlorine, is preferred.

The chemical promoter can be diluted with a gas which is inert toward the dissociation reaction, with the use of hydrogen chloride being preferred. The amount of inert gas used as diluent should not exceed 5 mol % of the feed stream.

The intensity of the electromagnetic radiation or the particle beam or the amount of the chemical promoter is set so that the molar conversion, based on the feed, at the dissociation gas-end outlet of the feed vaporizer 40 is in the range from 50 to 65%, preferably from 52 to 57%.

Particular preference is given to a molar conversion, based on the EDC used, at the dissociation gas-end outlet of the feed vaporizer 40 of 55%.

The temperature of the reaction mixture leaving the reactor 20 is lowered compared with conventional processes and is in the range from 400° C. to 470° C.

The process of the invention is particularly preferably used for the thermal dissociation of 1,2-dichloroethane to form vinyl chloride.

The process of the invention includes not only the thermal dissociation of halogenated, aliphatic hydrocarbons in the actual dissociation furnace 20 but also, as further process step, the vaporization of the liquid feed, for example the liquid EDC, before entry into the radiation zone 16 of the dissociation furnace 20. These measures have to be taken into account together with the actual thermal dissociation or the operation of the dissociation furnace 20 in order to determine the economics of the dissociation process.

A preferred embodiment of the invention is directed to a process in which the sensible heat of the dissociation gas is exploited in order to vaporize liquid, preheated feed, e.g. EDC, before entry into the radiation zone 16, preferably using a heat exchanger 40 as has already been described in EP 276,775 A2. Particular attention should here be given to ensuring that firstly the dissociation gas is still hot enough on leaving the dissociation furnace 20 to vaporize the total amount of the feed by means of its sensible heat content and secondly the temperature of the dissociation gas on entering this heat exchanger 40 does not go below a minimum value in order to prevent condensation of tar-like substances in the heat exchanger tubes.

In a further preferred embodiment of the vaporization of the feed, which has likewise been described in EP 276,775 A2, the temperature of the dissociation gas at the exit from the dissociation furnace 20 is so low that the heat content of the dissociation gas is not sufficient to vaporize the feed completely. In this embodiment of the invention, the missing proportion of gaseous feed is produced by flash evaporation of liquid feed in a vessel 54, preferably in the steaming-out vessel of a heat exchanger 40, as has been described in EP 276,775 A2. In this case, preheating of the liquid feed advantageously occurs in the convection zone 17 of the dissociation furnace 20. In this embodiment of the invention, too, it has to be ensured that the temperature of the dissociation gas at the inlet into this heat exchanger 40 does not go below a minimum value in order to prevent condensation of tar-like substances in the heat exchanger tubes 22a, 22s, 22b.

In a preferred embodiment of the process of the invention, the heat content of the dissociation gas is used to vaporize at least 80% of the feed by means of indirect heat exchange without the dissociation gas condensing either partly or completely.

As heat exchanger, preference is given to using an apparatus as is described, for example, in EP 264,065 A1. Here, liquid halogenated aliphatic hydrocarbon is heated indirectly by the hot product gas comprising the ethylenically unsaturated halogenated hydrocarbon which leaves the reactor 20, vaporized and the resulting gaseous feed gas is introduced into the reactor 20, with the liquid halogenated aliphatic hydrocarbon being heated to boiling by the product gas in a first vessel 52 and from there being transferred to a second vessel 54 in which it is partly vaporized without further heating under a pressure which is lower than in the first vessel 52 and the vaporized feed gas being fed into the reactor 20 and the unvaporized halogenated aliphatic hydrocarbon being recirculated to the first vessel 52.

In a particularly preferred variant of this process, the halogenated aliphatic hydrocarbon is heated in the convection zone 17 of the reactor 20 by means of the flue gas 38 produced by the burners 26 which heat the reactor 20 before being fed into the second vessel 54.

Particular preference is given to a mode of operation in which the entire feed is vaporized by indirect heat exchange with the dissociation gas without the dissociation gas being either partly or completely condensed.

If the feed is not vaporized completely by means of the heat content of the dissociation gas, the residual amount of feed is preferably vaporized by flash evaporation into a vessel 54, with the feed being preheated beforehand in the liquid state in the convection zone 17 of the dissociation furnace 20. As vessel for the flash evaporation, preference is given to using the steaming-out vessel 54 of a heat exchanger 40, as has been described, for example, in EP 264,065 A1.

In a further preferred process variant, the molar conversion of the dissociation reaction is determined downstream of the point at which the dissociation gas leaves the EDC vaporizer or at the top of the quenching column, for example by means of an on-line analytical apparatus, preferably by means of an on-line gas chromatograph GC.

The amount of fuel can be divided in either unequal parts or preferably equal parts over the burner rows 26 in the furnace 20.

The economics of the process are also influenced by the sum of the pressure drops over the dissociation furnace 20 (comprising convection zone 17 and radiation zone 16), the heat exchanger 40 for vaporization of the feed and also any quenching system (“quenching column”) present. This should be as low as possible since when the dissociation products are separated off by distillation, they have to be condensed at the top of a column using a refrigeration machine for cooling the condenser. The greater the sum of the pressure drops over the total system for “thermal dissociation”, the lower the pressure of the top of the column and the dissociation product separated off, for example HCl, has to be condensed at a correspondingly lower temperature. This leads to an increased specific energy consumption by the refrigeration machine, which in turn has an adverse effect on the economics of the total process.

1-30. (canceled)

31. An improved process for the thermal dissociation of halogenated aliphatic hydrocarbons to form ethylenically unsaturated halogenated hydrocarbons in a reactor which comprises reaction tubes running through a convection zone and through a radiation zone located downstream in the flow direction of the reaction gas, which has one or more burners in the radiation zone to heat the reaction tubes, and a heating apparatus is provided for the halogenated aliphatic hydrocarbon which is located outside the reactor and is heated by the energy content of the reaction gases leaving the radiation zone, wherein the improvement comprises:
a.) introducing a controlled input of an initiator for the thermal dissociation of said halogenated aliphatic hydrocarbon into the reaction tubes at one or more points within the reactor, said initiator being chosen from the group consisting of at least one chemical promoter for the thermal dissociation reaction; a localized energy input adapted to form free radicals to promote the thermal dissociation reaction; and combinations of the foregoing:
b.) controlling the input of an initiator and amount of the fuel for the burners such that:
i.) the conversion of the dissociation reaction changes by no more than 20%, if at all, compared to operation without the chemical promoter and/or the localized energy input,
ii.) the temperature of the reaction mixture leaving the reactor is in the range from 400° C. to 470° C., and
iii.) the molar conversion based on the halogenated aliphatic hydrocarbon used is in the range from 50 to 65%.

32. The process as claimed in claim 31, wherein the dew point of the flue gas is determined downstream from the exit from the convection zone and the input of initiator and amount of fuel for the burners is controlled in response thereto.

33. The process as claimed in claim 31, wherein the a heat exchanger is provided through which flue gas may be passed, and wherein said flue gas is condensed in said heat exchanger and the heat of the flue gas is utilized to preheat air supplied to the burner.

34. The process as claimed in claim 33, wherein the heat exchange is effected at the exit of the flue gas from the convection zone.

35. The process as claimed in claim 34, wherein:
a.) flue gas from the reactor is condensed in a heat exchanger and heat derived therefrom is utilized to preheat air fed to the burners; and
b.) the dew point of the flue gas at the exit from the convection zone or in the flue gas chimney is determined and the input of initiator and amount of fuel for the burners is controlled in response thereto.

36. The process as claimed in claim 31, wherein the localized energy input to form free radicals is effected by means of electromagnetic radiation or by means of a particle beam.

37. The process as claimed in claim 36, wherein the electromagnetic radiation is ultraviolet laser light.

38. The process as claimed in claim 31, wherein elemental chlorine is used as chemical promoter.

39. The process as claimed in claim 38, wherein the elemental chlorine is diluted with hydrogen chloride, with the amount of the hydrogen chloride used for dilution being not more than 5 mol % of the halogenated aliphatic hydrocarbon stream used.

40. The process as claimed in claim 31, wherein the molar conversion based on the halogenated aliphatic hydrocarbon used is in the range from 52 to 57%.

41. The process as claimed in claim 40, wherein the halogenated aliphatic hydrocarbon is 1,2-dichloroethane and the ethylenically unsaturated halogenated hydrocarbon is vinyl chloride.

42. The process as claimed in claim 31, wherein the halogenated aliphatic hydrocarbon is 1,2-dichloroethane and the ethylenically unsaturated halogenated hydrocarbon is vinyl chloride.

43. The process as claimed in claim 42, wherein liquid halogenated aliphatic hydrocarbon fed to the reactor is indirectly heated and vaporized by the hot product gas comprising ethylenically unsaturated halogenated hydrocarbon leaving the reactor, and the resulting gaseous feed gas is re-introduced into the reactor, with the liquid halogenated aliphatic hydrocarbon being heated to boiling by the hot product gas in a first vessel and from there being transferred to a second vessel in which it is partly vaporized without further heating under a pressure which is lower than in the first vessel and the vaporized feed gas is fed back to the reactor and unvaporized halogenated aliphatic hydrocarbon is recirculated to the first vessel.

44. The process as claimed in claim 31, wherein liquid halogenated aliphatic hydrocarbon fed to the reactor is indirectly heated and vaporized by the hot product gas comprising ethylenically unsaturated halogenated hydrocarbon leaving the reactor, and the resulting gaseous feed gas is re-introduced into the reactor, with the liquid halogenated aliphatic hydrocarbon being heated to boiling by the hot product gas in a first vessel and from there being transferred to a second vessel in which it is partly vaporized without further heating under a pressure which is lower than in the first vessel and the vaporized feed gas is fed back to the reactor and unvaporized halogenated aliphatic hydrocarbon is recirculated to the first vessel.

45. The process as claimed in claim 44, wherein the halogenated aliphatic hydrocarbon is heated by flue gas produced by the burners which heat the reactor in the convection zone of the reactor before the heated halogenated aliphatic hydrocarbon is fed to the second vessel.

46. The process as claimed in claim 43, wherein said reactor has an interior wall and further comprises a flue gas chimney having an interior wall, wherein:
a command temperature is measured at one or more points chosen from either
the interior wall temperature of the flue gas chimney;
the interior wall temperature of the dissociation furnace at the exit of the flue gas from the coldest flue-gas-side section of the convection zone and
the input of fuel to the burners and input of initiator is regulated in response thereto.

47. The process as claimed in claim 31, wherein said reactor has an interior wall and further comprises a flue gas chimney having an interior wall, wherein:
a command temperature is measured at one or more points chosen from either
the interior wall temperature of the flue gas chimney;
the interior wall temperature of the dissociation furnace at the exit of the flue gas from the coldest flue-gas-side section of the convection zone and
the input of fuel to the burners and input of initiator is regulated in response thereto.

48. The process as claimed in claim 31, wherein the flue gas chimney is thermally insulated.

49. The process as claimed in claim 48, wherein the flue gas chimney is trace-heated.

50. The process as claimed in claim 49, wherein a chimney blower is provided and the flue gas is extracted from the dissociation furnace thereby.

51. The process as claimed in claim 33, wherein:
a flue gas blower is provided; and
the flue gas:
is extracted by means of said flue gas blower after leaving the convection zone and
is condensed by being passed through one or more heat exchangers, and
the heat obtained thereby is utilized for heating air fed to the burner.

52. The process as claimed in claim 51, wherein the flue gas:
a.) is introduced in a downward direction from above into the heat exchanger;
b.) is cooled to below the dew point of the flue gas therein; and,
c.) after cooling, leaves the heat exchanger in the upward direction; and
the condensate formed thereby runs off downward from the heat exchanger.

53. The process as claimed in claim 31, wherein:
a.) the halogenated aliphatic hydrocarbon is 1,2-dichloroethane;
the ethylenically unsaturated halogenated hydrocarbon is vinyl chloride;
b.) the molar conversion based on the 1,2-dichloroethane used is in the range from 52 to 57%;
c.) the liquid 1,2-dichloroethane fed to the reactor is indirectly heated and vaporized by the hot product gas comprising vinyl chloride leaving the reactor, and the resulting gaseous feed gas is re-introduced into the reactor, with the liquid 1,2-dichloroethane being heated to boiling by the hot product gas in a first vessel and from there being transferred to a second vessel in which it is partly vaporized without further heating under a pressure which is lower than in the first vessel and the vaporized feed gas is fed back to the reactor and unvaporized 1,2-dichloroethane is recirculated to the first vessel;
d.) flue gas from the reactor is condensed in a heat exchanger and heat derived therefrom is utilized to preheat air fed to the burners; and
e.) the dew point of the flue gas downstream from the convection zone is determined and the input of initiator and amount of fuel for the burners is controlled in response thereto.

54. The process as claimed in claim 53, wherein said reactor has an interior wall and further comprises a flue gas chimney having an interior wall, wherein:
a command temperature is measured at one or more points chosen from either
the interior wall temperature of the flue gas chimney;
the interior wall temperature of the dissociation furnace at the exit of the flue gas from the coldest flue-gas-side section of the convection zone and
the input of fuel to the burners and input of initiator is regulated in response thereto.

55. The process as claimed in claim 54, wherein the flue gas chimney is thermally insulated.

56. The process as claimed in claim 54, wherein the flue gas chimney is trace-heated.

57. The process as claimed in claim 56, wherein a chimney blower is provided and the flue gas is extracted from the dissociation furnace thereby.

58. The process as claimed in claim 53, wherein:
a flue gas blower is provided; and
the flue gas:
is extracted by means of said flue gas blower after leaving the convection zone and
is condensed by being passed through one or more heat exchangers, and
the heat obtained thereby is utilized for heating air fed to the burner.

59. The process as claimed in claim 58, wherein the flue gas:
a.) is introduced in a downward direction from above into the heat exchanger;
b.) is cooled to below the dew point of the flue gas therein; and,
c.) after cooling, leaves the heat exchanger in the upward direction; and
the condensate formed thereby runs off downward from the heat exchanger.

60. An improved apparatus for the thermal dissociation of halogenated aliphatic hydrocarbons to form ethylenically unsaturated halogenated hydrocarbons, which comprises a reactor having:
a.) a convection zone; and a radiation zone defined therein,
b.) reaction tubes running through the convection zone and through the radiation zone, the radiation zone being located downstream in the flow direction of the reaction gas,
c.) at least one burner being provided in the radiation zone to heat the reaction tubes,
d.) a flue gas chimney; and
said apparatus further comprising a heating apparatus for the halogenated aliphatic hydrocarbon which is:
i.) located outside the reactor; and
ii.) heated by the energy content of the reaction gases leaving the radiation zone,
wherein the improvement comprises:
e.) means for introducing an initiator for accelerating the dissociation of said halogenated aliphatic hydrocarbon including:
i.) means of introducing chemical promoters for the thermal dissociation reaction into the reaction tubes;
ii.) means of introducing localized energy to form free radicals in the reaction tubes, or
111.) a combination of means for introducing chemical promoters and localized energy to form free radicals;
f.) means of for evaluating the degree of dissociation of said halogenated aliphatic hydrocarbon by:
i.) determining the dew point of the flue gas at the exit from the convection zone; means of introducing the fuel for the burner(s),
ii.) determining the dew point of the flue gas in the flue gas chimney, or
iii.) determining the molar conversion of the dissociation reaction, or
iv.) a combination of any of the foregoing i.), ii.) and iii.); and
g.) means for controlling the degree of dissociation of said aliphatic halogenated hydrocarbon by:
i.) regulating the amount of fuel;
ii.) regulating the input of initiator; or
iii.) a combination of the foregoing i.) and ii.) in response to:
iv.) the dew point of the flue gas at the exit from the convection zone; or
v.) the dew point of the flue gas in the flue gas chimney; or
vi.) the molar conversion of the dissociation reaction.

61. The apparatus as claimed in claim 60, wherein the improvement further comprises at least one heat exchanger adapted to recover heat from the flue gas by condensation thereof and thereby preheating combustion air for the burners.

62. The apparatus as claimed in claim 61, wherein the means of determining the molar conversion of the dissociation reaction is mounted downstream of the exit of the dissociation gas from the heating apparatus for the halogenated aliphatic hydrocarbon.

63. The apparatus as claimed in claim 60, wherein the means of determining the molar conversion of the dissociation reaction is mounted downstream of the exit of the dissociation gas from the heating apparatus for the halogenated aliphatic hydrocarbon

64. The apparatus as claimed in claim 60, wherein the means of introducing chemical promoters for the thermal dissociation together with the halogenated aliphatic hydrocarbon into the reaction tubes in the radiation zone are feed lines which allow the introduction of predetermined amounts of chemical promoters into the feed gas stream.

65. The apparatus as claimed in claim 60, wherein the means of introducing chemical promoters for the thermal dissociation at one or more points in the radiation zone into the reaction tubes are feed lines which allow the introduction of predetermined amounts of chemical promoters into the reaction tubes at the level of the radiation zone and open into the reaction tubes in the first third, viewed in the flow direction of the reaction gas, of the radiation zone.

66. The apparatus as claimed in claim 60, wherein the means of introducing localized energy to form free radicals in the reaction tubes at one or more points in the radiation zone are chosen from the group consisting of:
feed lines via which a plasma may be introduced into the reaction tubes at the level of the radiation zone; or
windows via which electromagnetic radiation may be injected into the reaction tubes at the level of the radiation zone,
said means of introducing localized energy to form free radicals in the reaction tubes being installed in the tubes in the first third, viewed in the flow direction of the reaction gas, of the radiation zone.

67. The apparatus as claimed in claim 60, wherein the heating apparatus for the halogenated aliphatic hydrocarbon located outside the reactor comprises a first vessel and a second vessel, with the liquid halogenated aliphatic hydrocarbon being heated to boiling by the product gas in the first vessel and from there being transferred to the second vessel in which it is partly vaporized without further heating under a pressure which is lower than in the first vessel with the vaporized feed gas being fed into the reactor and the unvaporized halogenated aliphatic hydrocarbon being recirculated to the first vessel.

68. The apparatus as claimed in claim 67, wherein the halogenated aliphatic hydrocarbon is conveyed in a pipe through the convection zone of the reactor where it is heated by means of the flue gas produced by the burners which heat the reactor before being fed into the second vessel.

69. The apparatus as claimed in claim 60, wherein the flue gas chimney is thermally insulated.

70. The apparatus as claimed in claim 69, wherein the flue gas chimney is trace-heated.

71. The apparatus as claimed in claim 60, wherein at least one chimney blower downstream from the exit from the convection zone for extracting the flue gas from the dissociation oven.
